(19) United States
(12) Patent Application Publication
(10) Pub. No.: US 2015/0119975 A1
(43) Pub. Date: Apr. 30, 2015

(54) BRANCHED VESSEL PROSTHESIS FOR REPAIR OF A FAILED STENT GRAFT

(71) Applicant: THE CLEVELAND CLINIC FOUNDATION, Cleveland, OH (US)

(72) Inventor: Tara M. Mastracci, Cleveland, OH (US)

(21) Appl. No.: 14/522,778

(22) Filed: Oct. 24, 2014

Related U.S. Application Data
(60) Provisional application No. 61/894,978, filed on Oct. 24, 2013.

(51) Int. Cl. A61F 2/856

(52) U.S. Cl. CPC A61F 2/856
USPC 623/1.35

(57) ABSTRACT

One aspect of the present disclosure relates to a branched vessel prosthesis for repair of a failed stent graft. The branched vessel prosthesis can include a main stent graft body having a proximal end portion, a distal end portion, and a frusto-conical midsection that tapers to the distal end portion. The main stent graft body can include a tubular wall that defines a main lumen. The midsection can include a fenestration in the tubular wall and a collapsible internal limb that extends from the fenestration into the main lumen. The internal limb can be configured to receive an iliac limb extension.

### RELATED APPLICATIONS

This application claims the benefit of U.S. Provisional Patent Application Ser. No. 61/894,978, filed Oct. 24, 2013, the entirety of which is hereby incorporated by reference for all purposes.

### TECHNICAL FIELD

The present disclosure relates generally to medical devices and, more particularly, to endovascular prostheses, systems, and methods for repair of failed stent grafts, such as failed abdominal aortic stent grafts.

### BACKGROUND

An infra-renal abdominal aortic aneurysm (AAA) is a localized dilation of the abdominal aorta below the renal arteries. The majority of patients with AAAs are asymptomatic, with the aneurysm detected only on routine examination, or in the course of investigations for other conditions. However, rupture is the most common complication of AAA, the risk of which increases with aneurysm diameter such that when an aneurysm reaches 5.5 cm, surgical intervention is considered.

Only two surgical treatments are routinely used in practice for treatment of infra-renal AAAs—infra-renal endovascular aneurysm repair (IREVAR) and open surgical repair (OSR). IREVAR is a minimally-invasive surgical technique that involves two small cuts in the groin (rather than an incision along the full length of the abdominal wall), and insertion of a stent graft prosthesis through the femoral artery. When compared with OSR, IREVAR is associated with marked reduction in post-operative morbidity, a decrease in mortality, and a quicker return to pre-operative health status, in addition to quality of life benefits. A significant portion of IREVAR devices fail, however, and endovascular conversion to fenestrated repair requires a long sealing zone, which can be constrained by IREVAR devices with short bodies.

### SUMMARY

The present disclosure relates generally to medical devices and, more particularly, to endovascular prostheses, systems, and methods for repair of failed stent grafts, such as failed abdominal aortic stent grafts.

One aspect of the present disclosure relates to a branched vessel prosthesis for repair of a failed stent graft. The branched vessel prosthesis can include a main stent graft body having a proximal end portion, a distal end portion, and a frusto-conical midsection that tapers to the distal end portion. The main stent graft body can include a tubular wall that defines a main lumen. The midsection can include a fenestration in the tubular wall and a collapsible internal limb that extends from the fenestration into the main lumen. The internal limb can be configured to receive an iliac limb extension.

Another aspect of the present disclosure can relate to a system for uni-iliac repair of a failed stent graft. The system can comprise a branched vessel prosthesis and an aorto-uni-iliac converter sized and dimensioned for placement within the branched vessel prosthesis. The branched vessel prosthesis can include a main stent graft body having a proximal end portion, a distal end portion, and a frusto-conical midsection that tapers to the distal end portion. The main stent graft body can include a tubular wall that defines a main lumen. The midsection further includes a fenestration in the tubular wall and a collapsible internal limb that extends from the fenestration into the main lumen. The internal limb can be configured to receive an iliac limb extension. The aorto-uni-iliac converter can comprise a main body having a proximal end portion, a distal end portion, and a frusto-conical midsection that tapers to the distal end portion. The midsection of the aorto-uni-iliac converter can include a surface configured to sealingly occlude blood flow through the fenestration when the aorto-uni-iliac converter is inserted into the branched vessel prosthesis.

Another aspect of the present disclosure can relate to a method for repairing a failed stent graft. One step of the method can include providing a branched vessel prosthesis. The branched vessel prosthesis can include a main stent graft body having a proximal end portion, a distal end portion, and a frusto-conical midsection that tapers to the distal end portion. The main stent graft body can include a tubular wall that defines a main lumen. The midsection can further include a fenestration in the tubular wall and a collapsible internal limb that extends from the fenestration into the main lumen. The internal limb can be configured to receive an iliac limb extension. Next, the branched vessel prosthesis can be inserted into the failed stent graft. A distal end portion of a branch stent graft can then be inserted through the fenestration into the internal limb so that the main lumen, a lumen of the internal limb, and a lumen of the branch stent graft are in fluid communication with one another.

Another aspect of the present disclosure can relate to a method for uni-iliac repair of a failed stent graft. One step of the method can include providing a branched vessel prosthesis. The branched vessel prosthesis can include a main stent graft body having a proximal end portion, a distal end portion, and a frusto-conical midsection that tapers to the distal end portion. The main stent graft body can include a tubular wall that defines a main lumen. The midsection can further include a fenestration in the tubular wall and a collapsible internal limb that extends from the fenestration into the main lumen. Next, the branched vessel prosthesis can be inserted into the failed stent graft. An aorto-uni-iliac converter can then be inserted into the branched vessel prosthesis. The aorto-uni-iliac converter can comprise a main body having a proximal end portion, a distal end portion, and a frusto-conical midsection that tapers to the distal end portion. After inserting the aorto-uni-iliac converter into the branched vessel prosthesis, the aorto-uni-iliac converter can be secured in the branched vessel prosthesis so that a surface of the midsection of the aorto-uni-iliac converter sealingly occludes blood flow through the fenestration.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1A is a schematic illustration showing a front view of a branched vessel prosthesis for repair of a failed stent graft constructed in accordance with one aspect of the present disclosure;

FIG. 1B is a schematic illustration showing a side view of the branched vessel prosthesis in FIG. 1A;

FIG. 1C is a top view of the branched vessel prosthesis in FIGS. 1A-B;

FIG. 1D is a schematic illustration showing a back view of the branched vessel prosthesis in FIGS. 1A-C;

FIG. 1E is a cross-sectional view taken along Line 1E-1E in FIG. 1D;

FIG. 2A is a schematic illustration showing a side view of an aorto-uni-iliac converter constructed in accordance with another aspect of the present disclosure;

FIG. 2B is a schematic illustration showing a back view of the aorto-uni-iliac converter in FIG. 2A;

FIG. 2C is a top view of the aorto-uni-iliac converter shown in FIGS. 2A-B;

FIG. 3 is a process flow diagram illustrating a method for repairing a failed stent graft according to another aspect of the present disclosure;

FIG. 4 is a schematic illustration showing a failed short body stent graft;

FIG. 5 is a schematic illustration showing the branched vessel prosthesis (FIGS. 1A-C) inserted in the failed short body stent graft of FIG. 4;

FIG. 6 is a schematic illustration showing an iliac limb extension being inserted into a collapsible internal limb of the branched vessel prosthesis in FIG. 5;

FIG. 7 is a process flow diagram illustrating a method for uni-iliac repair of a failed stent graft according to another aspect of the present disclosure; and

FIG. 8 is a schematic illustration showing a system comprising an aorto-uni-iliac converter (FIGS. 2A-C) and a branched vessel prosthesis (FIGS. 1A-C) implanted in a failed short body stent graft.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

### Overview

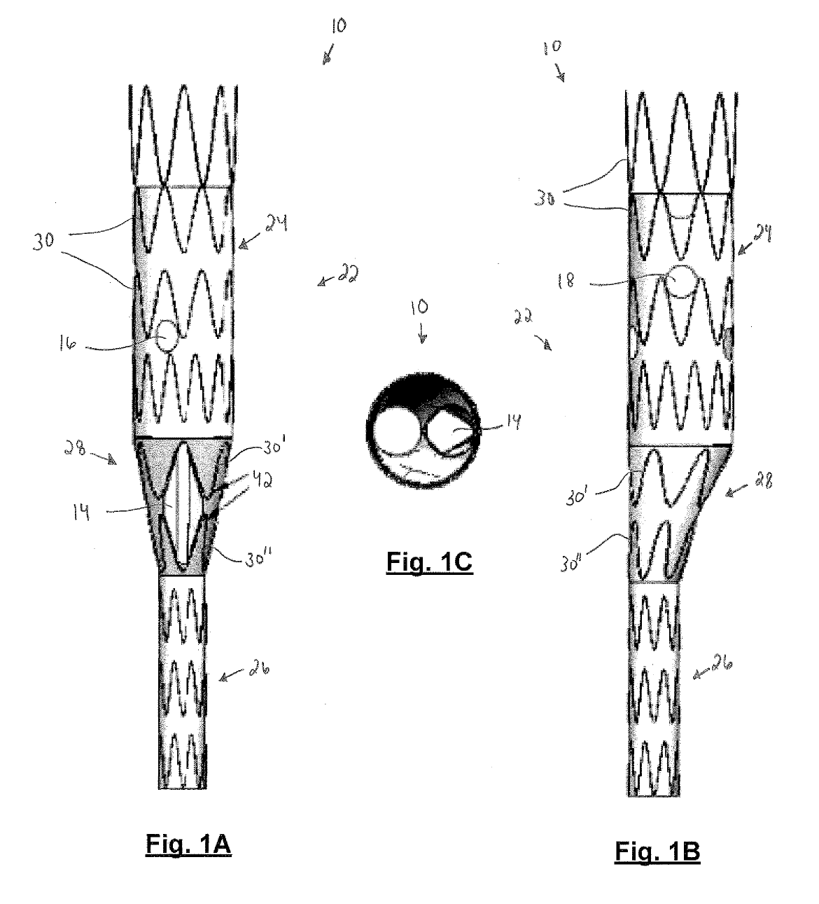

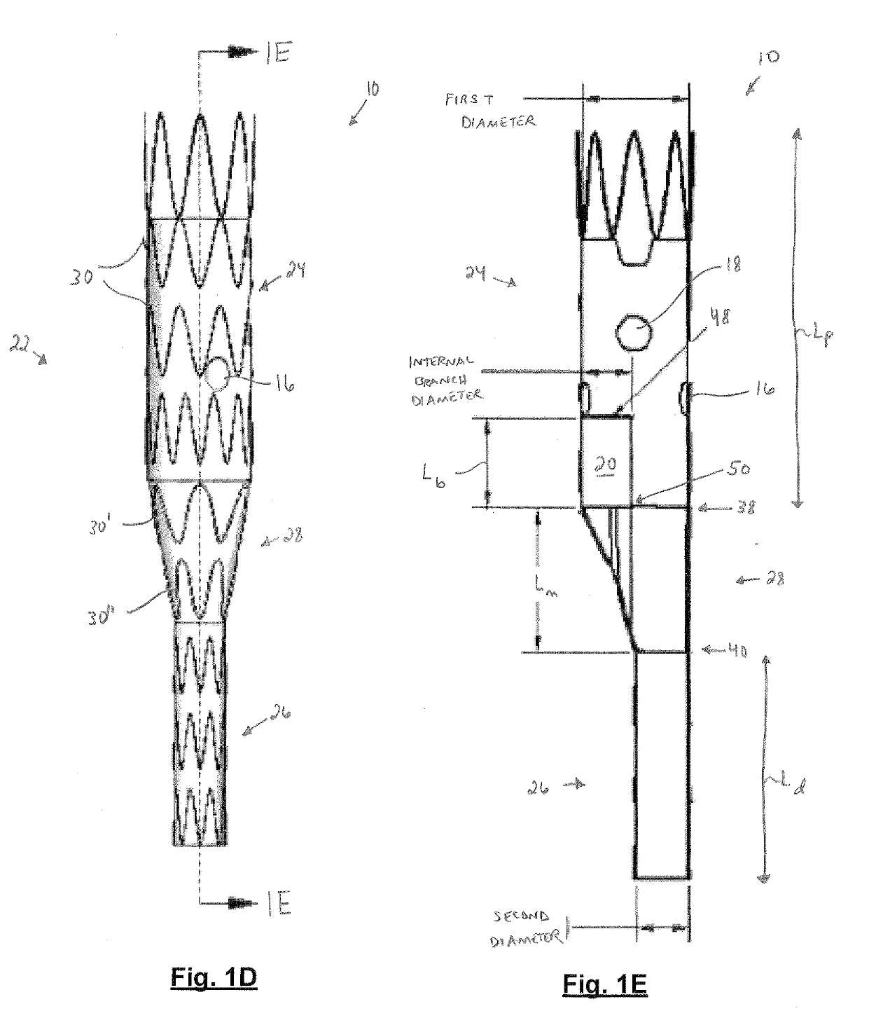

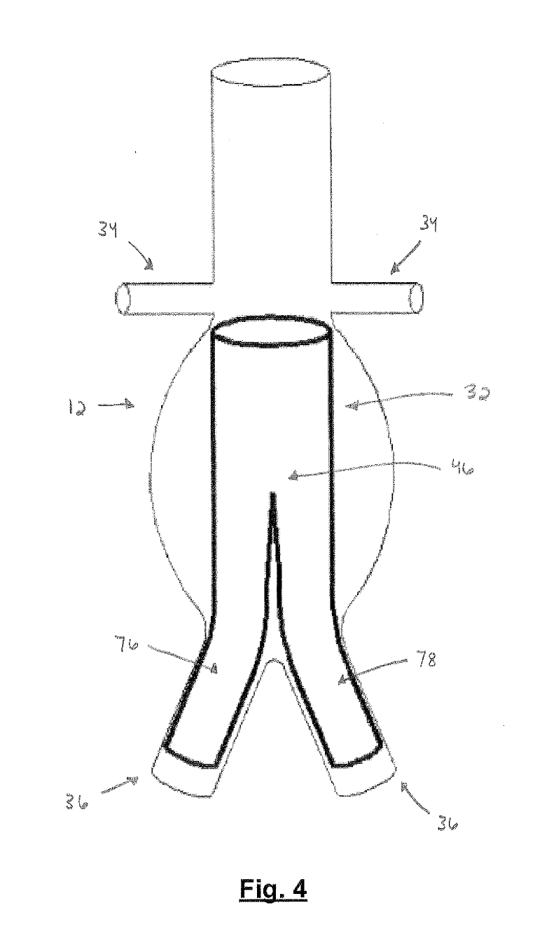

The present disclosure relates generally to medical devices and, more particularly, to endovascular prostheses, systems, and methods for repair of failed stent grafts, such as failed abdominal aortic stent grafts. As shown in FIGS. 1A-E, one aspect of the present disclosure can include a branched vessel prosthesis 10 for repair of a failed stent graft, such a failed long body stent graft (not shown) or a failed short body stent graft 12 (FIG. 4). Abdominal aortic aneurysm repair has undergone a revolution since endoluminal repair was first described in the early 1990s. Subsequent data from large registries have confirmed its efficacy. Despite the promise of endoluminal repair for infra-renal aneurysms, for example, a significant portion of infra-renal endovascular aneurysm repair (IREVAR) devices fail. One method of rescue is fenestrated conversion. Endovascular conversion to fenestrated repair requires a long sealing zone, which can be constrained by IREVAR devices with short bodies or, if long-bodied, such devices are tortuous. As discussed in more detail below, the present disclosure advantageously provides a branched vessel prosthesis 10 (FIGS. 1A-E) configured to shorten the required sealing zone and thereby increase the number of failed IREVAR which can be rescued. The branched vessel prosthesis 10 of the present disclosure can include any number of fenestrations 14, 16 and 18, which are customizable to suit patient anatomy or standard pivot fenestrations. Additionally, the branched vessel prosthesis 10 can include a collapsible or crushable internal limb 20, which may be used for branch access, iliac limb placement, or may collapse for uni-iliac indications. These features of the present disclosure, as well as others discussed herein, make the branched vessel prosthesis 10 an off-the-shelf option for endovascular conversion to fenestrated repair of failed stent grafts.

### Branched Vessel Prosthesis

One aspect of the present disclosure can include a branched vessel prosthesis 10. The branched vessel prosthesis 10 can be sized and dimensioned for repair of a failed short body stent graft, such as a failed long body stent graft or a failed short body stent graft 12 (FIG. 4). The branched vessel prosthesis 10 (FIGS. 1A-E) can be manufactured as an off-the-shelf item adapted to sealingly fit within a failed stent graft. In some instances, the branched vessel prosthesis 10 can be constructed in a similar or identical manner as conventional stent grafts, which are generally made of a stent and a graft material associated therewith. For example, a stent graft can be a tubular device with walls made from a flexible sheet material, supported by a rigidizing frame, which is usually formed from super-elastic metal. Some stent graft designs can be fixed to a vessel wall by means of barbs or hooks. The frame maintains the tubular shape of the stent graft while providing a radial sealing force to create a proximal and distal seal with a vessel wall. Examples of materials that can be used to form stent grafts are known.

In another aspect, the branched vessel prosthesis 10 can comprise a main stent graft body 22 having a proximal end portion 24, a distal end portion 26, and a frusto-conical midsection 28 that tapers to the distal end portion. The main stent graft body 22 can be made of a biocompatible graft material and a plurality of Z stents 30 that are sutured to the inside and/or outside of the graft material. The main stent graft body 22 includes a tubular wall that defines a main lumen. It will be appreciated that the diameter and/or length of each component of the main stent graft body 22 (i.e., the proximal end portion 24, the distal end portion 26, and/or the midsection 28) can be sized and dimensioned to accommodate patient-specific vascular anatomy.

The proximal end portion 24 of the main stent graft body 22 includes a first diameter that can be customized based on a patient's vascular anatomy. The proximal end portion 24 of the main stent graft body 22 is adapted for insertion into a main body of a failed stent graft. For example, the proximal end portion 24 of the main stent graft body 22 can be adapted for insertion into a main body 32 (FIG. 4) of a failed short body stent graft 12. In some instances, the proximal end portion 24 (FIGS. 1A-E) is sized and dimensioned such that placement of the branched vessel prosthesis 10 into a failed stent graft 12 results in an outer circumferential surface of the proximal end portion being in fluid tight contact with an inner circumferential surface of the main body of the failed stent graft. The first diameter of the proximal end portion 24 can be the same or substantially the same along the length Lₚ of the proximal end portion. In some instances, the proximal end portion 24 can include a plurality of fenestrations 16 and 18. For example, a plurality of secondary fenestrations 16 can be located about the proximal end portion 24 so that the secondary fenestrations are radially aligned with the renal arteries 34 (FIG. 4) when the branched vessel prosthesis 10 (FIGS. 1A-E) is implanted in a patient. In other instances, the proximal end portion 24 can additionally or optionally include one or more fenestrations 18 configured to be a pivot branch (P branch) (not shown).

The distal end portion 26 of the main stent graft body 22 has a tubular configuration and extends distally from the midsection 28. The distal end portion 26 has a second diameter that, in some instances, can be about 12 Fr. In other instances, the second diameter can be less than the first diameter of the proximal end portion 24. The distal end portion 26 can be sized and dimensioned for insertion into an iliac artery 36 (FIG. 4). The distal end portion 26 (FIGS. 1A-E) also includes a length L_{d}, which can be varied as needed.

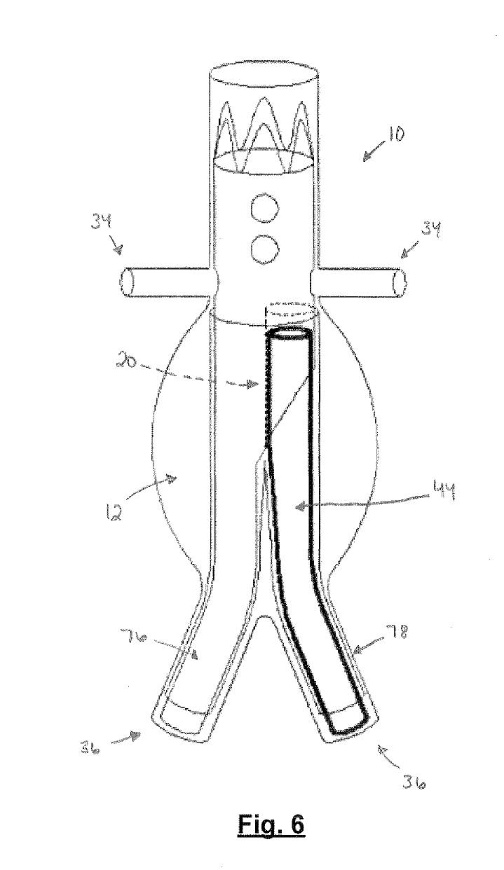

The midsection 28 of the branched vessel prosthesis 10 extends between the proximal and distal end portions 24 and 26 of the main stent graft body 22. The midsection 28 has a frusto-conical shape and a diameter that tapers along a length Lₘ of the midsection. In one example, the length Lₘ of the midsection 28 can be between about 20 mm to about 40 mm (e.g., 34 mm). In some instances, the diameter of the midsection 28 at a first end 38 can be equal, or about equal to, the first diameter, while the diameter of the midsection at a second end 40 can be equal, or about equal to, the second diameter. The midsection 28 can include first and second Z stents 30′ and 30″, each of which has a plurality of oppositely disposed points 42. As shown in FIG. 1A, the first and second Z stents 30′ and 30″ are arranged about the midsection 28 in a point-to-point configuration to define a diamond-shaped fenestration 14. The point-to-point configuration permits formation of a relatively large fenestration 14, which advantageously promotes attachment of an iliac limb extension 44 (FIG. 6) to the branched vessel prosthesis 10 (FIGS. 1A-E). It will be appreciated that the dimensions of the fenestration 14 can be tailored to a patient's anatomy by, for example, selecting first and second Z stents 30′ and 30″ having a desired size and/or shape. As described in more detail below, the midsection 28 is adapted to fit snugly at a bifurcation of a failed stent graft 12 so that the fenestration 14 is in fluid communication with a lumen of an iliac artery 36 (FIG. 4).

As shown in FIG. 1E, the midsection 28 includes a collapsible or crushable internal limb 20 that extends from the fenestration 14 into the main lumen. The internal limb 20 can have a tubular or substantially tubular shape. The internal limb 20 can be made of a flexible material (e.g., ePTFE) capable of being readily expanded and collapsed. The internal limb 20 can include a tubular wall, which, when expanded, forms a lumen that extends between oppositely disposed first and second ends 48 and 50. In some instances, the internal limb 20 can be physically free of any stents (e.g., Z-stents or zig zag stents) or other similar metallic support structures.

The internal limb 20 can have a length L_{b} of about 20 mm (e.g., at least about 20 mm) and a diameter that is equal, or about equal to, the second diameter of the distal end portion 26. In one example, the distance between the middle of the fenestration 14 to the second end 48 of the internal limb 20 can be about 35 mm. In some instances, the internal limb 20 can be located about 1 mm to about 6 mm (e.g., 3 mm) distal to the fenestration 14. The internal limb 20 can be configured to receive a portion of an iliac limb extension 44 (FIG. 6) so that the main lumen of the main stent graft body 22 (FIGS. 1A-E) is in fluid communication with a lumen of the iliac limb extension. As discussed in more detail below, the collapsible and expandable properties of the internal limb 20 advantageously allow the branched vessel prosthesis 10 to be used for branch access, iliac limb placement, or can collapse to convert to uni-iliac indications. It will be appreciated that the internal limb 20 can include one or more flexible rings (not shown) to maintain the patency of the lumen when the internal limb is expanded. In one example, a Nitinol ring can be disposed at the first end 48 of the internal limb 20.

### System

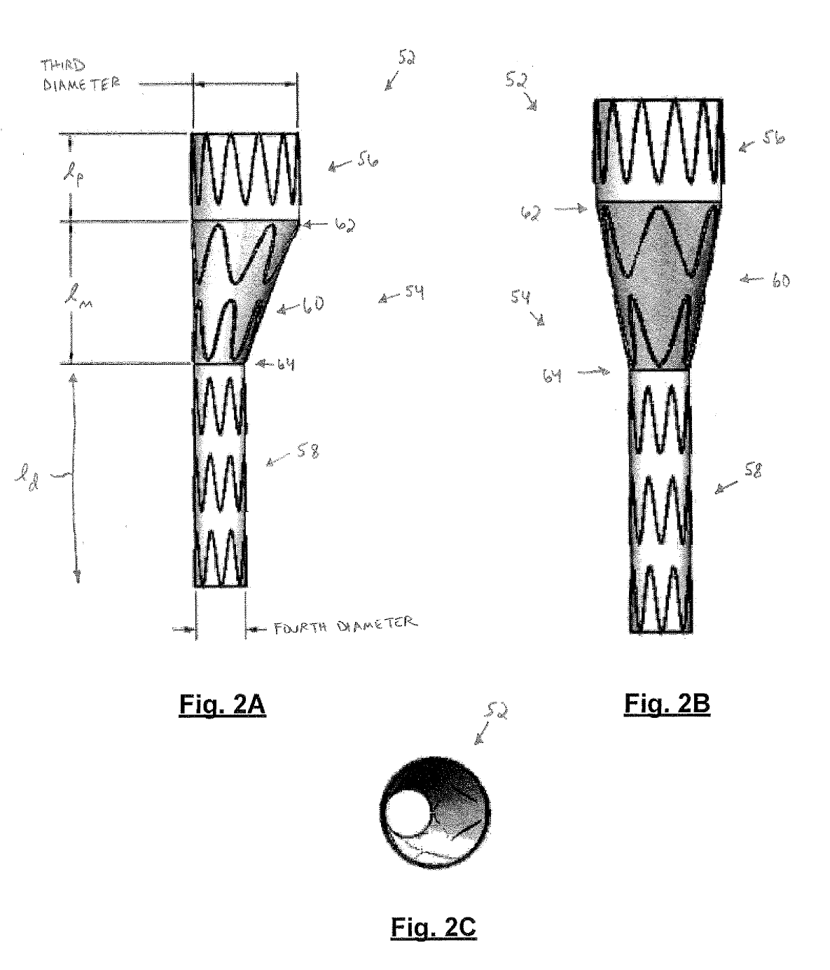

Another aspect of the present disclosure includes a system for uni-iliac repair of a failed stent graft, such as a failed abdominal aortic stent graft 12. The system can include a branched vessel prosthesis 10 (FIGS. 1A-E) and an aorto-uni-iliac converter 52 (FIGS. 2A-C), which is sized and dimensioned for placement within the branched vessel prosthesis (FIGS. 1A-E). The aorto-uni-iliac converter 52 (FIGS. 2A-C) can be constructed in a similar or identical manner as the branched vessel prosthesis 10 (FIGS. 1A-E). For example, the aorto-uni-iliac converter 52 (FIGS. 2A-C) can be comprised of a biocompatible graft material and a plurality of Z stents 30 that are sutured to the inside and/or outside of the graft material. As shown in FIGS. 2A-C, the aorto-uni-iliac converter 52 can comprise a main body 54 having a proximal end portion 56, a distal end portion 58, and a frusto-conical midsection 60 that tapers to the distal end portion. As described in more detail below, the dimensions of the proximal end portion 56, the distal end portion 58, and the midsection 60 are substantially the same as the corresponding dimensions of the branched vessel prosthesis 10 (FIGS. 1A-E) so long as the aorto-uni-iliac converter 52 (FIGS. 2A-C) is insertable into the branched vessel prosthesis to form a fluid tight seal therebetween.

The proximal end portion 56 of the aorto-uni-iliac converter 52 can include a third diameter that can be customized based on a patient's vascular anatomy. The proximal end portion 56 is adapted for insertion into a portion of the branched vessel prosthesis 10 (FIGS. 1A-E). For example, the proximal end portion 56 (FIGS. 2A-C) can be sized and dimensioned such that placement of the aorto-uni-iliac converter 52 within the branched vessel prosthesis 10 (FIGS. 1A-E) results in an outer circumferential surface of the proximal end portion (FIGS. 2A-C) being in fluid tight contact with an inner circumferential surface of the proximal end portion 24 of the branched vessel prosthesis. The third diameter of the proximal end portion 56 (FIGS. 2A-C) can be the same or substantially the same (e.g., less than) along a length lₚ of the proximal end portion. In some instances, the length lₚ of the proximal end portion 56 can be equal to or less than the length Lₚ (FIGS. 1A-E) of the proximal end portion 24 of the branched vessel prosthesis 10. For example, the length lₚ (FIGS. 2A-C) of the proximal end portion 56 can be between about 15 mm to about 25 mm. In another example, the length lₚ of the proximal end portion 56 is no more than 21 mm.

The distal end portion 58 of the aorto-uni-iliac converter 52 has a tubular configuration and extends distally from the midsection 60. The distal end portion 58 has a fourth diameter that, in some instances, can be the same as, or slightly less than, the second diameter of the distal end portion 26 (FIGS. 1A-E) of the branched vessel prosthesis 10 (e.g., about 12 Fr). In other instances, the fourth diameter can be less than the third diameter of the proximal end portion 56 (FIGS. 2A-C). The distal end portion 58 can be sized and dimensioned for insertion into the distal end portion 26 (FIGS. 1A-E) of the branched vessel prosthesis 10. The distal end portion 58 (FIGS. 2A-C) also includes a length l_{d}, which can be less than, equal to, or greater than the length L_{d} of the distal end portion 26 (FIGS. 1A-E) of the branched vessel prosthesis 10.

The midsection 60 (FIGS. 2A-C) of the aorto-uni-iliac converter 52 extends between the proximal and distal end portions 56 and 58 of the main body 54. The midsection 60 has a frusto-conical shape and a diameter that tapers along a length lₘ of the midsection. In one example, the length lₘ of the midsection 60 can be equal to, or less than, the length Lₘ of the midsection of the branched vessel prosthesis 10 (FIGS. 1A-E) (e.g., 34 mm). In some instances, the diameter of the midsection 60 (FIGS. 2A-C) at a first end 62 can be equal, or about equal, to the third diameter, while the diameter of the midsection at a second end 64 can be equal, or about equal, to the fourth diameter. The midsection 60 can include first and second Z stents 30′ and 30″, each of which has a plurality of oppositely disposed points 42. In some instances, the first and second Z stents 30′ and 30″ are arranged about the midsection 60 in a point-to-point configuration. Unlike the midsection 28 (FIGS. 1A-E) of the branched vessel prosthesis 10, the midsection 60 (FIGS. 2A-C) of the aorto-uni-iliac converter 52 does not include a fenestration 14. Rather, the tubular wall defining the midsection 60 is a continuous surface, at least a portion of which is sized and dimensioned to sealingly occlude blood flow through the fenestration 14 (FIGS. 1A-E) when the aorto-uni-iliac converter 52 (FIGS. 2A-C) is inserted into the branched vessel prosthesis 10 (FIGS. 1A-E).

### Methods

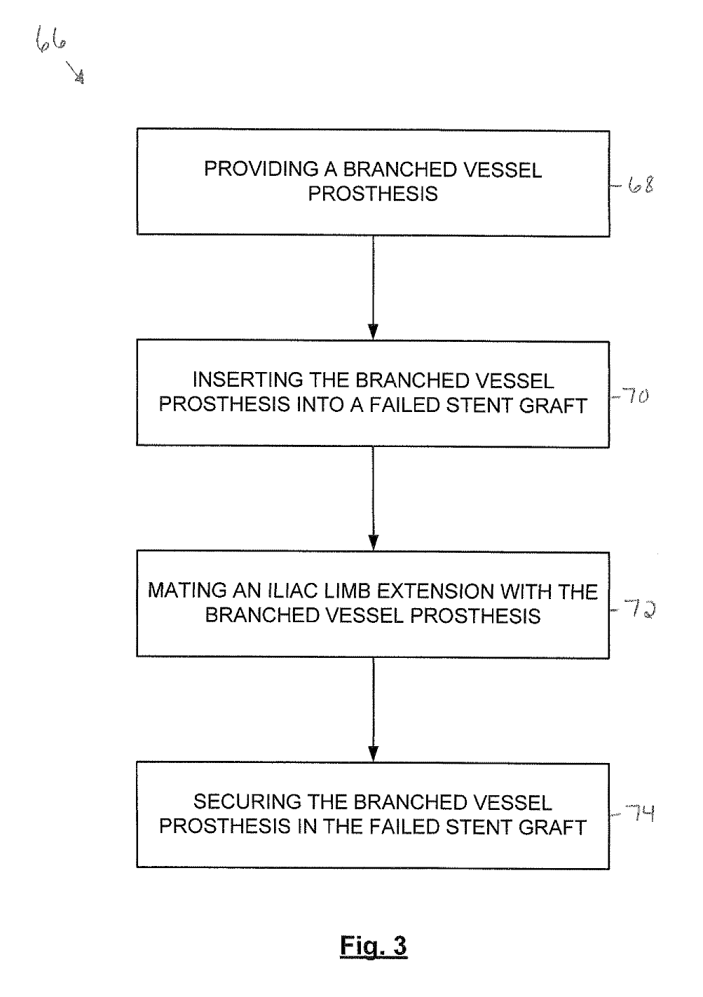

Another aspect of the present disclosure includes a method 66 (FIG. 3) for repairing a failed stent graft. As shown in FIG. 3, the method 66 can generally include the steps of providing a branched vessel prosthesis (Step 68), inserting the branched vessel prosthesis into a failed stent graft (Step 70), mating an iliac limb extension with the branched vessel prosthesis (Step 72), and securing the branched vessel prosthesis in the failed stent graft (Step 74). The method 66 is described below in terms of repairing a failed short body stent graft 12 (FIG. 4). One skilled in the art will appreciate that the method 66 (FIG. 3) can be used to treat any type of failed stent graft, such as a failed abdominal aortic stent graft (e.g., a failed long body stent graft).

As shown in FIG. 4, a failed short body stent graft 12 can generally include a bifurcation 46 formed by first and second legs 76 and 78. IREVAR devices, such as the short body stent graft 12 shown in FIG. 4, will often fail if an adequate seal is not formed between the luminal wall of the abdominal aorta and the outer surface of the IREVAR device. The infra-renal/aortic neck region is particularly unstable as this region will dilate, leak, and potentially rupture unless it is protected by a securely fixed stent graft. As discussed below, the method 66 (FIG. 3) advantageously enables endovascular conversion to fenestrated repair of failed short body stent grafts 12 (FIG. 4) by combining the main body 32 and bifurcation 46 of the failed short body stent graft to create an elongated sealing zone that prevents dilation and leakage at the infra-renal/aortic neck region.

At Step 68 of the method 66, a branched vessel prosthesis 10 can be provided. The branched vessel prosthesis 10 can be constructed in a similar or identical manner as the one shown in FIGS. 1A-E and described above. The branched vessel prosthesis 10 can be sized and dimensioned to accommodate specific patient anatomy. Prior to insertion into the patient, for example, measurements of the patient's abdominal aorta (e.g., at the infra-renal/aortic region) can be obtained using one or a combination of imaging modalities. After obtaining the measurements, the dimensions of the branched vessel prosthesis 10 can be tailored accordingly.

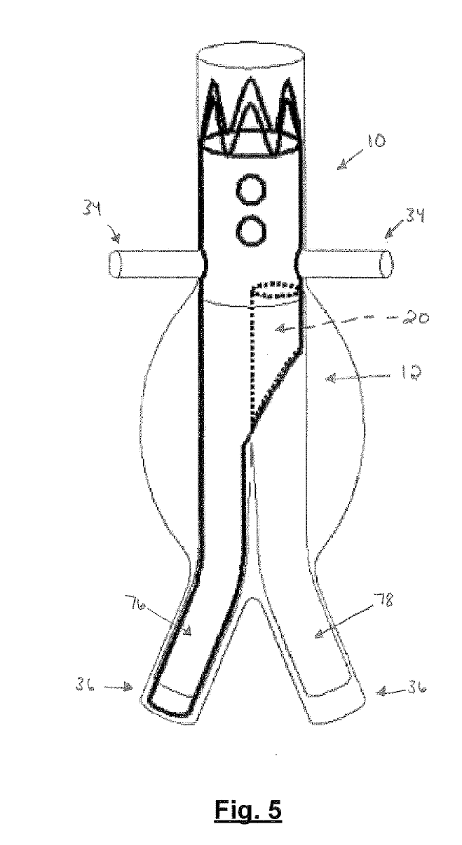

Once an appropriately-sized branched vessel prosthesis 10 has been selected, the branched vessel prosthesis can be inserted into the failed short body stent graft 12 (Step 70). In some instances, the branched vessel prosthesis 10 can be loaded into an endovascular delivery system (not shown). The endovascular delivery system can then be positioned adjacent the failed short body stent graft 12, whereafter the branched vessel prosthesis 10 can be deployed into the failed short body stent graft. As shown in FIG. 5, for example, the branched vessel prosthesis 10 can be inserted into the short body stent graft 12 so that the distal end portion 26 is received in the first leg 76 of the failed short body stent graft, the midsection 28 is seated at the bifurcation 46, and the proximal end portion 24 is received within the main body 32. With the branched vessel prosthesis 10 disposed in the failed short body stent graft 12, the secondary fenestrations 16 of the proximal end portion 24 can be radially aligned with the renal arteries 34. Additionally, the proximal end portion 24 of the main stent graft body 22 can extend beyond the proximal end of the failed short body stent graft 12 to form a fluid tight seal with the vessel wall. At Step 72, the branched vessel prosthesis 10 can be secured within the failed short body stent graft 12 by virtue of the fluid tight seal formed therebetween and/or placement of sutures (or other fasteners).

Next, an iliac limb extension 44 (FIG. 6) can be mated with the branched vessel prosthesis 10 at Step 74. To do so, the iliac limb extension 44 can be loaded into a delivery catheter (not shown) and then inserted into the vasculature of the patient. The delivery catheter can be advanced to an iliac artery 36 where it is positioned adjacent the second leg 78 of the failed short body stent graft 12. Next, the iliac limb extension 44 can be deployed from the delivery catheter into the second leg 78. The iliac limb extension 44 can then be progressively advanced until its distal end portion is received within the lumen of the internal limb 20. The distal end portion of the iliac limb extension 44 can be friction fit and/or secured by means of fasteners (e.g., sutures) within the internal limb 20. With the iliac limb extension 44 securely connected to the branched vessel prosthesis 10, the main lumen of the main stent graft body 22, the lumen of the internal limb 20, and a lumen of the iliac limb extension 44 can be in fluid communication with one another such that blood can flow through the branched vessel prosthesis 10 and thereby safely bypass the infra-renal aneurysm.

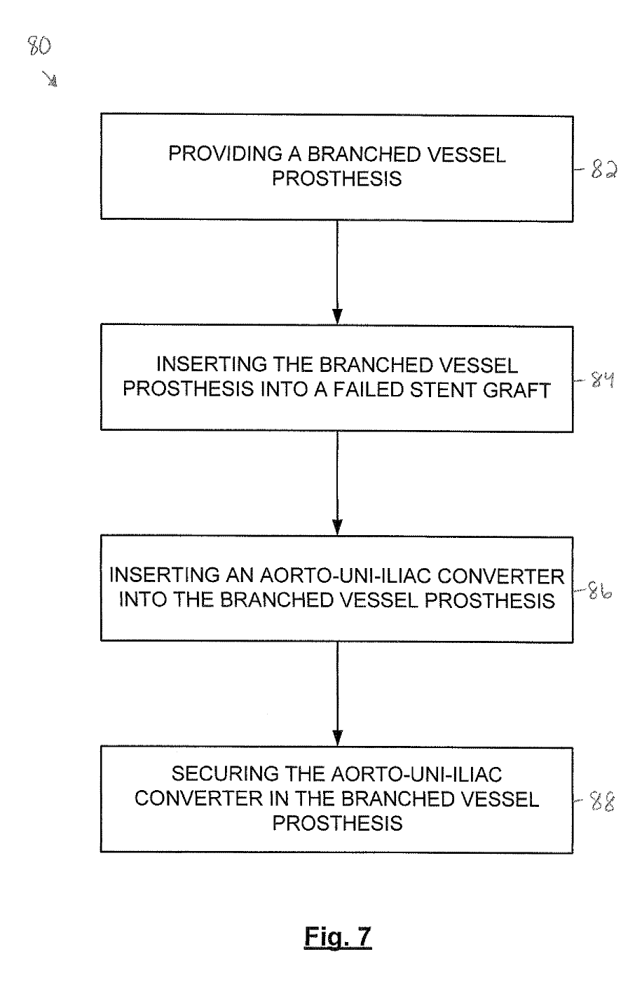

Another aspect of the present disclosure includes a method 80 (FIG. 7) for uni-iliac repair of a failed stent graft. As shown in FIG. 7, the method 80 can generally include the steps of: providing a branched vessel prosthesis (Step 82); inserting the branched vessel prosthesis into a failed stent graft (Step 84); inserting an aorto-uni-iliac converter into the branched vessel prosthesis (Step 86); and securing the aorto-uni-iliac converter in the branched vessel prosthesis (Step 88). Steps 82 and 84 of the method 80 can be performed in an identical or substantially identical manner as described for Steps 68 and 80 (FIG. 3). As discussed above, it will be appreciated that although the method 80 is described below in terms of treating a failed short body stent graft 12 (FIG. 4), the method can find use in treating a variety of failed stent grafts, such as a failed abdominal aortic stent graft (e.g., a failed long body stent graft).

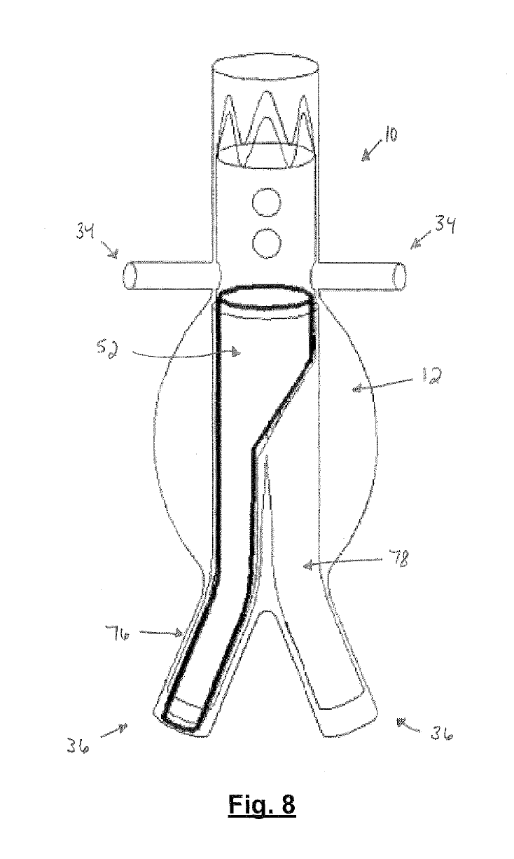

Once a branched vessel prosthesis 10 has been inserted into a failed short body stent graft 12, the aorto-uni-iliac converter 52 (FIG. 8) can be inserted into the branched vessel prosthesis at Step 86. The aorto-uni-iliac converter 52 can be identically or similarly constructed as the aorto-uni-iliac converter shown in FIGS. 2A-C and described above. In some instances, the aorto-uni-iliac converter 52 can be pre-loaded into the branched vessel prosthesis 10 so that the aorto-uni-iliac converter and the branched vessel prosthesis are deliverable to the failed short body stent graft 12 using a single endovascular delivery device. Advantageously, such an approach makes only single iliac access necessary.

With the aorto-uni-iliac converter 52 disposed in the branched vessel prosthesis 10, the internal limb can 20 obtains its collapsed configuration and thereby allow the midsection 60 of the aorto-uni-iliac converter to be seated at the bifurcation 46 of the failed short body stent graft 12. The midsection 60 of the aorto-uni-iliac converter 52 then provides a surface that sealingly covers or occludes the fenestration 14 of the branched vessel prosthesis 10 so as to prevent blood flow through the second leg 78 of the failed short body stent graft 12. Since blood flow to the corresponding iliac artery 36 is blocked by the aorto-uni-iliac converter 52, an arterial bypass procedure can be performed so that blood can flow from the abdominal aorta, around the failed short body stent graft 12, and into the iliac artery.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

The following is claimed:

1. A branched vessel prosthesis for repair of a failed stent graft, the branched vessel prosthesis comprising:
a main stent graft body having a proximal end portion, a distal end portion, and a frusto-conical midsection that tapers to the distal end portion, the main stent graft body including a tubular wall that defines a main lumen;
wherein the midsection includes a fenestration in the tubular wall and a collapsible internal limb that extends from the fenestration into the main lumen, the internal limb being configured to receive an iliac limb extension.

2. The branched vessel prosthesis of claim 1, wherein the fenestration is diamond-shaped.

3. The branched vessel prosthesis of claim 1, wherein the proximal end portion of the main stent graft body includes a plurality of secondary fenestrations, each of the secondary fenestrations being configured to receive a pivot branch device.

4. The branched vessel prosthesis of claim 1, wherein the failed stent graft is a failed short body stent graft or a failed long body stent graft.

5. The branched vessel prosthesis of claim 1, wherein the proximal end portion of the main stent graft body is adapted for insertion to a main body of the failed stent graft.

6. The branched vessel prosthesis of claim 1, wherein the proximal end portion of the main stent graft body is sized and dimensioned such that placement of the branched vessel prosthesis into the failed stent graft results in an outer circumferential surface of the proximal end portion being in fluid tight contact with an inner circumferential surface of the main body of the failed stent graft.

7. The branched vessel prosthesis of claim 1, wherein a plurality of secondary fenestrations is located about the proximal end portion of the main stent graft body so that the secondary fenestrations are radially aligned with the renal arteries when the branched vessel prosthesis is implanted in a patient.

8. The branched vessel prosthesis of claim 1, wherein the proximal end portion of the main graft stent body has a second diameter that is less than a first diameter.

9. The branched vessel prosthesis of claim 1, wherein the internal limb has a diameter that is equal, or about equal to, a second diameter of the distal end portion.

10. A system for uni-iliac repair of a failed short body stent graft, the system comprising:
a branched vessel prosthesis that includes a main stent graft body having a proximal end portion, a distal end portion, and a frusto-conical midsection that tapers to the distal end portion, the main stent graft body including a tubular wall that defines a main lumen, the midsection further including a fenestration in the tubular wall and a collapsible internal limb that extends from the fenestration into the main lumen, the internal limb being configured to receive an iliac limb extension; and
an aorto-uni-iliac converter that is sized and dimensioned for placement within the branched vessel prosthesis, the aorto-uni-iliac converter comprising a main body having a proximal end portion, a distal end portion, and a frusto-conical midsection that tapers to the distal end portion, the midsection including a surface configured to sealingly occlude blood flow through the fenestration when the aorto-uni-iliac converter is inserted into the branched vessel prosthesis.

11. The system of claim 10, wherein the fenestration is diamond-shaped.

12. The system of claim 10, wherein the proximal end portion of the main stent graft body includes a plurality of secondary fenestrations, each of the secondary fenestrations being configured to receive a pivot branch device.

13. The system of claim 10, wherein the failed stent graft is a failed short body stent graft or a failed long body stent graft.

14. The system of claim 10, wherein the proximal end portion of the main graft stent body is sized and dimensioned such that placement of the aorto-uni-iliac converter within the branched vessel prosthesis results in an outer circumferential surface of the proximal end portion being in fluid tight contact with an inner circumferential surface of the proximal end portion of the branched vessel prosthesis.

15. The system of claim 10, wherein the midsection of the aorto-uni-iliac converter does not include a fenestration.

16. A method for repairing a failed stent graft, the method comprising the steps of:
providing a branched vessel prosthesis that includes a main stent graft body having a proximal end portion, a distal end portion, and a frusto-conical midsection that tapers to the distal end portion, the main stent graft body including a tubular wall that defines a main lumen, the midsection further including a fenestration in the tubular wall and a collapsible internal limb that extends from the fenestration into the main lumen, the internal limb being configured to receive an iliac limb extension;
inserting the branched vessel prosthesis into the failed stent graft; and
inserting a distal end portion of an iliac limb extension through the fenestration into the internal limb so that the main lumen, a lumen of the internal limb, and a lumen of the iliac limb extension are in fluid communication with one another.

17. A method for uni-iliac repair of a failed stent graft, the method comprising the steps of:
providing a branched vessel prosthesis that includes a main stent graft body having a proximal end portion, a distal end portion, and a frusto-conical midsection that tapers to the distal end portion, the main stent graft body including a tubular wall that defines a main lumen, the midsection further including a fenestration in the tubular wall and a collapsible internal limb that extends from the fenestration into the main lumen;
inserting the branched vessel prosthesis into the failed stent graft;
inserting an aorto-uni-iliac converter into the branched vessel prosthesis, the aorto-uni-iliac converter comprising a main body having a proximal end portion, a distal end portion, and a frusto-conical midsection that tapers to the distal end portion; and
securing the aorto-uni-iliac converter in the branched vessel prosthesis so that a surface of the midsection of the aorto-uni-iliac converter sealingly occludes blood flow through the fenestration.
